(19) United States
(12) Patent Application Publication
(10) Pub. No.: US 2009/0028911 A1
(43) Pub. Date: Jan. 29, 2009

(54) PROCESS FOR THE PRODUCTION OF PIGMENT-BEARING LEADS

(75) Inventors: Reinhard Pinzer, Schnaittach (DE); Christian Sprogar, Bubenreuth (DE)

Correspondence Address: BACHMAN & LAPOINTE, P.C., 900 CHAPEL STREET, SUITE 1201, NEW HAVEN, CT, 06510, US

(73) Assignee: SCHWAN-STABILO COSMETICS GMBH & CO. KG, Heroldsberg (DE)

(21) Appl. No.: 11/576,948

(22) PCT Filed: Oct. 20, 2005
(86) PCT No.: PCT/EP2005/011313

§ 371 Date: May 14, 2007

(30) Foreign Application Priority Data
Oct. 22, 2004 (DE) 10 2004 051 554.9

(51) Int. Cl. A61K 8/18; B29C 47/00

(52) U.S. Cl. 424/401; 424/63; 264/239

(57) ABSTRACT

A process for the production of pigment-bearing and/or effect substance-bearing leads, comprising the following steps: a) pigments and/or effect substances are at least partially mixed with a fluid wetting agent so that a kneadable material is produced, b) further ingredients and optionally remaining pigments and/or effect substances are added and the resulting material optionally homogenised, c) the material obtained in b) is extruded to afford extrusions of predetermined diameter, and d) the extruded portions are dried to a constant weight optionally after adjustment of the extrusion length.

### BACKGROUND OF THE INVENTION

The invention concerns a process for the production of pigment-bearing and/or effect substance-bearing leads and leads which can be produced with that process for cosmetic pencils. The invention also concerns the use of such leads.

Pigment-bearing and/or effect substance-bearing leads are dry leads which are also referred to as powder leads, and can be used either in lead pencils or rotary pencils. For lead pencils, the lead is placed in a casing blank which is provided for same and which is then subjected to further processing in per se known manner to form a lead pencil. For a rotary pencil, the lead is fitted into a rotary mechanism. In both cases the pencil serves to apply aesthetic and coloured accents to skin and mucous membrane, in particular to the skin of the face, lips, eyelids and in the region of the eyes. In particular decorative cosmetics are considered as the main areas of use for pigmented powder leads. Pencils with powder leads are used in particular for eyeshadow, lipliner, eyeliner and eyebrow pencils. A further area of use are rouge pencils and concealer pencils. The leads used for those pencils are to be easy and pleasant to apply without scratching the sensitive skin, they are to adhere well after application to the place where they were applied and they are to be as transfer-resistant as possible, that is to say they are not to come out on to other surfaces and articles and they are not to bleed out of the region in which they were applied.

Those kinds of dry powder lead and various processes for the production thereof are known. In the case of the processes known from the state of the art, powder leads can either be produced by pressing a suitable and appropriately pretreated material into a shape or by extruding a material which is made into a paste in a suitable shape, cutting it to length and then drying it. For the sake of better handling those lead portions can be shrunk into a shrink tube. It is also possible to produce a suitable lead material using calcium sulphate hemihydrate (gypsum), make the material into a paste with water, and extrude it before it hardens. The last-mentioned process is less advantageous because the gypsum then very quickly and irreversibly hardens and then makes the material unusable.

Powder leads produced using the specified processes are generally glued into wood and are then offered in pencil form, but it is also known for such powder leads to be enclosed with a film sheathing which can be at least partially torn away, comprising paper, plastic or metal film or the like. Finally, there were also attempts to present such powder leads in a clamping casing sleeve, similarly to a lead pencil extension device.

References to the specified processes are to be found inter alia in DE-A 31 03 128, EP-A 0 057 266, DE-A 33 02 803, DE-A 33 47 756, DE-A 34 17 115 or DE-A 34 37 989. Processes are also known for the production of marbled powder leads and references thereto are to be found in U.S. Pat. No. 4,714,085. DE 31 03 128 and DE 35 35 999 describe processes for encasing a powder lead with a shrink tube. Leads of the specified kind were fitted into a suitable, sharpenable casing—any irregularities in the composition could be easily removed by sharpening. From the point of view of a customer however the operation of sharpening a cosmetic pencil is ultimately unsatisfactory as in that case a part of the makeup material is always unused and wasted.

Attempts were therefore undertaken to use leads of the specified kind in rotary mechanisms in order to use them in the manner known from lipsticks. The lead portions necessary for that purpose can be produced in the above-indicated manner and if necessary also shrunk in position. For that purpose the lead material is suitably made into a paste, extruded, dried and the resulting lead portion is then subjected to further processing. For extrusion of the material, particularly if the wish is to produce extrusions of a diameter in the range of 6 to 12 mm, high pressures in the range of 30 to 80 MPa are required. In that case, different flow speeds can occur within the lead material to be extruded, particularly when pearl pigments in flake form or also other flake-form, non-colouring ingredients such as for example transparent mica which is not coloured with metal oxides, synthetic mica, fine glass flakes or the like are used. Those pearl pigments and other flake-form ingredients can in that case also be oriented in a given manner and preferred direction, in accordance with the ‘law of least resistance’, and thereby provide zones which are structured in different ways, comprising the flake-form substances which are ‘oriented in the preferred direction’ and the other solid substances, within the lead. As moreover the flow speed of the material in the edge region of the tools necessary for the extrusion operation is lower than in the interior of the extrusion, in that way the edge regions can become very hard and support the interior, which then in itself is soft, of a portion of lead. Upon being applied to the skin, those hard edge zones of the lead portions produce a very unpleasant scratchy feel on the skin.

In addition those leads produce a non-homogeneous result as, depending on the respective viewing angle, apparently completely different colour effects occur by virtue of the preferred direction which is involved in terms of colour reflection and thus lead the lady consumer to believe that there is an apparently different and non-homogeneous material involved.

It is therefore necessary to seek such limit conditions which afford a visually homogeneous lead material, provide leads which as far as possible involve pearl flakes of different orientation over all regions and thus present a uniform coloration with a readily perceptible, intensive pearl sheen.

Therefore the object of the invention was to provide a lead material which has improved flow properties upon extrusion and which can be so extruded that a homogeneous lead is obtained. Another object of the invention was to provide leads which, in spite of a relatively large diameter for the lead extrusions, allow low pressures in the extrusion operation. A further object of the invention was to provide leads having a uniform surface, which allow lines to be drawn and/or permit application over an area and which at the same time have a pleasant feel on the skin.

### SUMMARY OF THE INVENTION

The object is achieved by providing a process for the production of pigment-bearing leads, which comprises the following steps:

a) pigments and effect substances are mixed with a fluid wetting agent so that a kneadable material is produced,

b) fillers and other further ingredients are added and the resulting material is optionally homogenised,

c) the material obtained in b) is extruded to afford extrusions of predetermined diameter, and

d) the extruded portions are dried to a constant weight optionally after adjustment of the extrusion length.

### DETAILED DESCRIPTION

The process according to the invention for the production of leads for a cosmetic pencil provides that pigments and effect substances are mixed with a fluid wetting agent so that the result is a kneadable material to which further ingredients are added. The resulting material is then homogenised, if necessary, and extruded to form extrusions of predetermined diameter. The extruded portions are then dried to a constant weight optionally after adjustment of the extrusion length.

It was surprisingly found that, when a process with specified steps a) to d) is used, leads with surprisingly advantageous properties are obtained. In particular the process according to the invention provides that the lead materials are extruded to afford very homogeneous leads which permit uniform application and which even after prolonged use do not form hard unpleasant edges.

The process according to the invention is suitable for all pigments and effect substances which are to be processed to provide powder leads, as are used in cosmetics. The terms pigments and effect substances are used here in the broadest sense to denote all substances which are incorporated into leads to produce a coloured, fluorescent, iridescent, light-reflecting impression and colourless pigments, particularly if they produce a special effect, are also embraced by those terms. Accordingly the term pigment in accordance with the present invention is intended to denote not only the conventional inorganic pigments but generally colourless, white or coloured inorganic or organic pigments, but also colour lakes, pearlescent agents and so-called ‘light diffusing pigments’ or ‘LDP’ and lakes of organic dyes. Metal powders and white or colourless powders such as mica, kaolin or talc can also be embraced thereby. The following can be mentioned by way of example for inorganic pigments, for example yellow, black or red iron oxides, titanium dioxide, zirconium oxide, cerium oxide, ultramarine, oxides of zinc and chromium, for example chromium oxide green, chromium hydroxide green, carbon black; organic pigments, lakes of organic pigments, carmine, flake-form metal powders such as passivated aluminium, brass, bronze, copper, silver or gold; mica, micas coated with metal oxides, for example with titanium dioxide, iron oxides, chromium oxide, chromium hydroxide; flake-form preparations based on silicon dioxide, aluminium oxide or glass, which can possibly also be coated with metal oxides, titanium dioxide, iron oxide, chromium oxide, chromium hydroxide and mixtures thereof. In accordance with the invention the term effect substances is used to denote all agents producing an aesthetic impression, for example luminescent, fluorescent, phosphorescent, iridescent, mother-of-pearl-like and thermochromic sub-stances, neon pigments, luminous pigments, interference pigments, metal flakes or spangles, colour-coated aluminium flakes or PET films, holographic elements, pearlescent agents and also UV-active dyes which in daylight and/or artificial lighting and/or with black light produce an effect. The terms pigments and effect substances overlap and are not deemed to be mutually exclusive.

In order to produce leads from pigments and/or effect substances, it is necessary to use binding agents, optionally fillers and thickening agents. All binding agents and fillers which are generally known for the production of cosmetic leads can be considered for production of the leads according to the invention. The binding agents and fillers serve to influence consistency and are used in dependence on the consistency desired for the lead material or the finished lead. The fillers or thickening agents for cosmetic leads that can be considered are for example natural gums and polymers such as xanthan gum, tragacanth and cellulose derivatives, synthetic polymers such as polyesters and polyolefins, inorganic substances such as clays, silicic acid, talc, kaolin, bentonite, hectorite, montmorrillonite, smectite, magnesium-aluminium-silicate, or metal soaps such as aluminium, magnesium, calcium or zinc stearate.

In order to obtain a homogeneous lead material, the incorporation of at least a part of the pigments and/or effect substances is so effected that they are processed with a wetting agent to afford a kneadable material. For that purpose at least a part of the pigments and/or effect substances is mixed with a fluid wetting agent in such a way that a kneadable material is produced, that kneadable material is then mixed with the remaining ingredients, optionally homogenised and then extruded and dried.

Preferably an inert wetting agent is used as the fluid wetting agent. The term ‘inert’ in accordance with the present invention denotes such a wetting agent which neither dissolves nor reacts with the pigments and/or effect substances and also binding agents and fillers and is also not absorbed. The inert agent serves to cause the particulate constituents—in particular pigments and effect substances—to slip past each other in the mixing operation without clinging to each other so that a homogeneous material can be formed. Suitable fluid wetting agents are in particular silicon-bearing compounds, for example siloxanes, and fluid hydrocarbons. Fluid wetting agents means in this context fluid at ambient temperature.

Preferred silicon-bearing wetting agents are compounds with repetitive R₂SiO_{2/2} units, wherein R is C₁-C₆ alkyl, cycloalkyl or aryl, preferably phenyl. Particularly preferred are those compounds with repetitive dimethylsiloxane units which are available in great varieties. Short-chain linear siloxanes and cyclic siloxanes which are also commercially available have proven to be particularly suitable. For example silicones made up from dimethylsiloxane units are available by the name dimethicone for linear siloxanes and cyclomethicone for cyclic siloxanes. The process and the leads of the present invention particularly preferably use linear dimethicones with 3 to 10 siloxane units, in particular 4 to 8 siloxane units, and cyclomethicones with 3 to 8, in particular 4 to 6 dimethylsiloxane units. The latter are commercially available under the names octamethylcyclotetrasiloxane, decamethylcyclopentasiloxane, and dodecamethylcyclohexasiloxane. Mixtures of those siloxanes are also suitable, which are also commercially available.

In particular paraffins and isoparaffins can be mentioned as fluid hydrocarbons. C₁₁-C₁₂ isoparaffin is a particularly suitable example. Short-chain monovalent alcohols such as ethanol, propan-1-ol, propan-2-ol, esters such as for example ethyl acetate, ketones such as for example acetone or methylethylketone or also mixtures of those liquids are also suitable so long as they are inert (as defined above) for the respective particulate constituents.

A further ingredient which is used according to the invention and which is preferably employed for the lead material according to the invention is a lubricant which adjusts the slip capability of the lead material during the extrusion operation. Both liquid and also solid agents are suitable as the lubricant, in which respect lubricants can act as a kind of lubricating agent while solid lubricants can perform the function of a ball bearing.

The materials usually employed for cosmetic materials can be used as lubricants for the lead according to the invention. Solid lubricants for the lead material according to the invention are inter alia fatty acid salts and boron nitrides in finely divided form such as for example fatty acid salts and boron nitride.

It has been found that particularly good results are achieved if fluorinated or phenylated siloxanes are added to the material as lubricant. Fluorinated and phenylated silicones or siloxanes are known per se and are commercially available. As an example mention may be made for example of perfluorononyldimethicone or methylphenyldimethicone. Copolymers of various siloxane units are also suitable for that purpose. As an example mention may be made of dimethicone/vinyldimethicone copolymer. Ethoxylated and/or propoxylated glycerides or glycerine partial esters have also proven satisfactory as a lubricant. PEG-20 glyceryl stearate can be mentioned here as a particularly suitable example. Mixtures of the specified constituents are also appropriate.

Tensides can also contribute to the lubricating action and are therefore used in preferred embodiments. Examples of suitable tensides are the cationic, anionic, non-ionic or amphoteric tensides which are usually used for cosmetic materials or mixtures thereof, preferably glycerides and glycerine partial fatty acid esters, but also esters of long-chain fatty acids and long-chain alcohols and further classes of compounds which are well-known to the man skilled in the art can be considered depending on the nature of the other ingredients.

The fillers that can be considered for cosmetic leads are in particular talc, kaolin and mica which at the same time can also act as pigments and/or effect substances.

Depending on the respective composition involved preserving agents can also be added to the leads. When dealing with water-free lead materials that is generally not necessary, while the usual ones are employed for water-bearing materials. Further usual ingredients for leads are antioxidants which improve storability and durability. An example in that respect is tocopherol derivatives. It is optionally also possible to use fragrances and aroma substances.

Frequently synthetic, mineral, animal or vegetable waxes are also used for adjusting consistency, such as paraffin, beeswax, carnauba and candelilla wax; oil raw materials such as hydrogenated vegetable oil or triglycerides, for example caprylic/capric triglycerides, and fats.

To improve workability the materials can contain moistening agents for keeping them moist, in the usual amounts. Examples in that respect are glycerine, 1,2-propylene glycol and 1,2-hexylene glycol.

To adjust consistency and to impart specific properties, synthetic or natural polymers are frequently also added to leads, which increase breaking and flexural strength, contribute to particularly good delivery properties and afford a pleasant feel on the skin. Examples of such polymers are carrageenan, tragacanth, alginate, carboxymethylcellulose, hydroxyethylcellulose, hydroxymethylcellulose as natural polymers and acrylates, methacrylates, polyurethanes, polyetherurethanes, polyesters and polyethers as synthetic polymers. Mixtures of individual natural polymers, individual synthetic polymers or natural and synthetic polymers can be used. The polymers are appropriately used in the form of aqueous dispersions to facilitate homogeneous distribution.

The leads according to the invention can be processed to provide concealer pencils and in addition to the constituents which have rather an aesthetic effect can also have therapeutic agents such as panthenol, salicylic acid or levulinic acid derivatives such as phenylethyl- and benzylesters, or other ingredients suitable for treating skin blemishes.

The pigments and/or effect substances or at least a part thereof are mixed with the fluid wetting agent, the fluid wetting agent being used in such an amount that the result is a plastic or kneadable material. The amount per se is not critical. If however the amount is too small, the material is difficult to work, and in particular difficult to extrude. If the proportion of the fluid wetting agent is too high, the properties of the finished lead are adversely affected. A proportion of 1 to 10% by weight, in particular 2 to 6% by weight, with respect to the weight of the lead material prior to extrusion is suitable. Mixing of pigments and effect substances with the wetting agent can be effected in a period of a few seconds to hours, for example within 10 seconds to about 60 minutes.

The further ingredients are then added to the kneadable material obtained in step a) of the process according to the invention and the resulting material is then optionally homogenised. Homogenisation is preferred in order to obtain a uniform material which results in a lead which is satisfactory aesthetically and in terms of its properties of use. Homogenisation is usually achieved in a period of a few seconds up to 120 minutes depending on the nature of the mixing apparatus used and the properties of the lead material. A period of 10 to about 300 seconds is generally sufficient.

The fillers and binding agents and optionally further lead ingredients which are used if required and optionally the rest of the pigments and/or effect substances are added to the kneadable material and intensively mixed. The ingredients involved in step b) can also be mixed with a fluid wetting agent to simplify the mixing procedure. For that purpose it is possible to use the same wetting agents as stated for step a), different agents therefrom and/or mixtures thereof.

Mixing of the constituents is effected in per se known mixing apparatuses, for example vibration mixers or ploughshare mixers. Homogenisation can then be effected in apparatuses which are also known per se and the man skilled in the art can select the homogeniser which is particularly suitable for the respective purpose.

The material obtained after steps a) and b) has flow properties which permit extrusion at lower pressures than is possible with the materials known in the state of the art. As a result the material also flows uniformly through the extrusion tool and supplies very uniform leads having mechanically advantageous properties. The homogenised material is therefore passed through an extrusion tool and extruded to form extrusions of predetermined diameter. The diameter of the extrusions depends on the respective purpose of use and can be adjusted by the man skilled in the art in per se known manner. The diameter depends on the one hand on the intended sleeve casing or rotary mechanism in which the lead is later to be fitted and on the other hand on the proportion of shrinkage which occurs due to subsequent drying. Usually the lead material is extruded to afford extrusions of a diameter in the range of 4 to 20 mm, in particular 6 to 12 mm. When dealing with leads of smaller diameter, the strength is generally too low to be able to use them in rotary mechanisms. Leads of a diameter larger than 20 mm are not preferred for the intended use. Leads of a diameter in the range of 6 to 12 mm have proven to be particularly suitable for the usual lead pencils and rotary mechanisms as they combine a particularly advantageous combination of breaking and flexural strength with application properties and delivery properties.

After the extrusion operation the extruded portions are usually cut to a desired extrusion length which is suitable for being laid in casing sleeves or fitted into rotary mechanisms. The corresponding pencil length depends on the respective casing sleeve or rotary mechanism and is known to the man skilled in the art. A suitable extrusion length is in the range of 20 to 100 mm, preferably 30 to 100 mm.

The extrusions are then dried to a constant weight. That drying operation to a constant weight is necessary to be able to manufacture reproducible leads. It has been found that, when carrying out the process according to the invention, leads of reproducible dimensions are obtained so that the process is suitable for lead manufacture. Drying to a constant weight, in particular with two drying sections in the preferred embodiment, provides leads of predeterminable and easily adjustable dimensions.

Extrusion of the lead material is effected in per se known manner in per se known extrusion tools. Tools and processes for same are numerous and are known to the man skilled in the art in this field. More detailed description in that respect is not required.

An essential feature of the process according to the invention is drying to a constant weight. The expression ‘drying to a constant weight’ is used to mean that the leads are dried until their weight no longer changes at a steady temperature for at least 5 minutes.

To carry out the drying operation, the extrusions which have possibly been cut to length are either exposed to a constant temperature for a sufficient period of time or preferably, they are exposed firstly to a lower temperature and then to a higher temperature, in two steps. If drying is effected at a constant temperature, temperatures in the range of 40 to 80° C. have proven to be suitable, which can be applied over a period of 1 to 24 hours. Particularly good properties are obtained for the leads when drying is effected firstly at a lower temperature and then at a higher temperature. In a particularly preferred procedure a vacuum is applied in the first drying section and then heating to a higher temperature is effected without a vacuum. Particularly good properties are achieved if, in the first stage, a temperature of about 40 to 80° C. is used and a vacuum of up to 80 kPa is applied. That first drying section can last for 5 minutes up to 12 hours and is preferably effected over a period of 10 to 120 minutes. In a second drying section drying is then effected without vacuum at a higher temperature to a constant weight, which can be checked in per se known manner by repeatedly taking samples and determining the weight thereof. Temperatures of the range of 80 to 150° C., in particular 90 to 120° C., have proven to be suitable temperatures for the second drying section. That second drying section is effected until a constant weight is reached, which is normally after 1 to 6 hours. The duration of that second drying treatment is not critical, the only essential point is that a constant weight is reached. Therefore the respectively appropriate drying period varies in dependence on the temperature used.

The advantages of the process according to the invention, that is to say reproducible, aesthetically and mechanically satisfactory leads, are achieved if at least a part of the pigments and/or effect substances is mixed with the fluid wetting agent and kneaded and drying of the leads is effected in the above-specified manner.

In the event that polysaccharides are used as fillers, the process according to the invention can also be supplemented by a further step which affords particularly good leads. In this specific embodiment a part of the pigments and/or effect sub-stances is not mixed with the fluid wetting agent, but mixed with an aqueous solution of the polysaccharide and a precipitation agent for the polysaccharide is added. The material produced in that way is then filtered so that the result is a plastic material with a residual moisture content in the range of up to 80%, preferably 40 to 60%. That material is then mixed together with the kneadable material obtained with the fluid wetting agent, and the further ingredients, and homogenised. The material produced in that way is then subjected to further processing in accordance with steps c) and d), as described above. This embodiment is particularly suitable if alginic acid or pectic acid is used as the binding agent, in which case precipitation is then preferably effected with a calcium solution.

A further subject of the invention is leads which can be obtained with the process according to the invention. Those leads are distinguished in relation to known leads in that they have a particularly homogenous and regular structure so that in use the applied colour or the applied effects remain the same.

A further subject of the invention is the use of the leads produced in accordance with the invention for the production of cosmetic pencils, in particular for producing eyeliners, lipliners, concealer and rouge pencils, eyebrow pencils, kohl and eyeshadow pencils.

The invention is further described by the Examples hereinafter.

The invention is to be described with reference to the following Examples, without this being definitive considerations in this respect. The raw materials used are in that respect denoted by the ‘INCI names’ which are known to the man skilled in the relevant art, and all amounts are stated in percent by weight (% by weight) with respect to the total weight of the extrudable paste.

### EXAMPLE 1

### Eyeshadow

| | | |
|---|---|---|
| (A) | Mica and titanium dioxide | 45.000 |
| | (C.I.-No. 77019, 77891) | |
| | Cyclopentasiloxane | 4.000 |
| | Phenoxyethanol | 0.500 |
| | Methylparaben | 0.150 |
| | Propylparaben | 0.050 |
| (B) | Dimethicone/vinyldimethicone copolymer | 2.000 |
| | Polyethylene (powder) | 5.000 |
| | Talc | 1.500 |
| | Kaolin | 3.500 |
| | Boron nitride | 3.500 |
| | Yellow iron oxide (C.I.-No. 77492) | 0.600 |
| | Red iron oxide (C.I.-No. 77491) | 1.900 |
| | Black iron oxide (C.I.-No. 77499) | 0.300 |
| (C) | Aqua | 34.400 |
| | Diethylhexyl sodium sulphosuccinate | 0.500 |
| | Hydroxyethylcellulose | 0.600 |

(A) The titanated mica is provided in a diaphragm vibration mixer. The parabens are then dissolved in phenoxyethanol, the cyclopentasiloxane is added to that mixture, then the total mixture is put into the diaphragm vibration mixer and the whole is intensively thoroughly mixed for about 3 minutes.

(C) The hydroxyethylcellulose is dissolved in water and then the tenside is added.

(B) In a ploughshare mixer the constituents of phase (B) are intensively thoroughly mixed, then the mixture is mixed with the previously mixed phase (C) and intensive mixing is effected for about 30 minutes. Thereafter the prepared phase (A) is added and the whole is thoroughly mixed once again for about 5 minutes.

The material is now extruded to form extrusions of a diameter of 8 mm and cut to length to provide portions 50 mm in length. Those portions now remain at ambient temperature for about 30 minutes and are then dried at 50° C. in a vacuum of 60 kPa. Drying is then effected to a constant weight without vacuum at 110° C. The result obtained is medium-brown, very shiny powder leads which can then be shrink-fitted into suitable shrink tubes.

### EXAMPLE 2

### Eyeshadow

| | | |
|---|---|---|
| (A) | Mica and titanium dioxide | 42.000 |
| | (C.I.-No. 77019, 77891) | |
| | Cyclopentasiloxane | 5.000 |
| | Perfluorononyl dimethicone | 1.750 |
| | Phenoxyethanol | 0.500 |
| | Methylparaben | 0.150 |
| | Propylparaben | 0.050 |
| | Carnauba (Copernicia Cerifera wax) | 0.500 |
| (B) | Yellow iron oxide (C.I.-No. 77492) | 0.450 |
| | Red iron oxide (C.I.-No. 77491) | 1.500 |
| | Black iron oxide (C.I.-No. 77499) | 0.150 |
| | Dimethicone/vinyldimethicone copolymer | 2.000 |
| | Polyethylene (powder) | 4.200 |
| | Talc | 1.500 |
| | Boron nitride | 3.300 |
| | Kaolin | 3.000 |
| | Silica | 0.800 |
| (C) | Aqua | 31.800 |
| | Ceteareth-30 | 0.650 |
| | Carboxymethylcellulose | 0.700 |

Production and processing are effected similarly to the method described in Example 1. The result obtained is a light-brown powder lead with a high sheen which can be very easily and uniformly distributed on the skin and affords good adhesion.

### EXAMPLE 3

### Eyeshadow

| | | |
|---|---|---|
| (A1) | Mica and titanium dioxide | 24.000 |
| | (C.I.-No. 77019, 77891) | |
| | Calcium chloride | 1.750 |
| | Aqua | 24.250 |
| (A2) | Alginic acid | 0.250 |
| | Aqua | 24.750 |
| (B) | Yellow iron oxide (C.I.-No. 77492) | 0.375 |
| | Red iron oxide (C.I.-No. 77491) | 0.900 |
| | Black iron oxide (C.I.-No. 77499) | 0.125 |
| | Dimethicone/vinyldimethicone copolymer | 1.000 |
| | Polyethylene (powder) | 2.250 |
| | Talc | 0.650 |
| | Boron nitride | 1.500 |
| | Kaolin | 1.250 |
| (C) | Diethylhexyl sodium sulphosuccinate | 0.250 |
| | PEG-40 hydrogenated castor oil | 0.175 |
| | Phenoxyethanol | 0.250 |
| | Methylparaben | 0.075 |
| | Propylparaben | 0.025 |
| | Aqua | 16.175 |

For production purposes the water from phase (A1) is provided in a vessel with propeller agitator, the calcium chloride is dissolved therein and the titanated mica is scattered thereon. Agitation is thereafter effected until a lump-free suspension is produced. In a separate vessel the alginic acid is dissolved in water with slight heating and agitation to about 50° C. and the mixture (A2) is added to the mixture (A1). Mixing is continued for about 30 minutes, followed by filtration, washing with a little water, and drying of the residue at 50° C. and under a vacuum of about 60 kPa to a constant weight, with granulation of that preparation.

The constituents of phase (B) are mixed in a ploughshare mixer, the preparation from phase (A) is added as a granulate and mixing is effected for about 20 minutes. In the meantime, in a separate vessel, the mixture of the constituents of phase (C) is produced with slight heating—initially without the addition of water—and then the water is added. That mixture of phases (A) and (B) is now transferred into a kneader, the aqueous phase (C) is added and mixing is effected for about 30 minutes until a homogeneous extrudable paste is produced.

Further procedure is now as described in the foregoing Examples. The result obtained is a markedly medium-brown coloured powder lead with a high sheen which can be easily and workably distributed on the skin and enjoys good adhesion.

### EXAMPLE 4

### Blusher

| | | |
|---|---|---|
| (A) | Mica and titanium dioxide | 18.000 |
| | (C.I.-No. 77019, 77891) | |
| | Mica (C.I.-No. 77019) | 22.000 |
| | Cyclopentasiloxane | 4.500 |
| | C11-12 isoparaffin | 1.500 |
| | Phenoxyethanol | 0.500 |
| | Methylparaben | 0.150 |
| | Propylparaben | 0.050 |
| (B) | Dimethicone/vinyldimethicone copolymer | 2.000 |
| | Polyethylene (powder) | 4.000 |
| | Talc | 1.000 |
| | Kaolin | 2.200 |
| | Boron nitride | 3.000 |
| | Titanium dioxide (C.I.-No. 77891) | 3.500 |
| | Red iron oxide (C.I.-No. 77491) | 3.000 |
| | Yellow iron oxide (C.I.-No. 77492) | 1.100 |
| | Black iron oxide (C.I.-No. 77499) | 0.200 |
| (C) | C11-12 isoparaffin | 27.700 |
| | Cyclopentasiloxane | 5.000 |
| | PEG-5 soya sterol | 0.600 |

Production is effected similarly to the above-described Examples 1 and 2, wherein the PEG-5 soya sterol is firstly dissolved with heating to 50 to 60° C. in the cyclopentasiloxane, mixed with the isoparaffin, and phase (C) is then added to the solids phase. The further procedure is then as described above. The lead portions which have been cut to length are pre-dried under an extractor hood which is in communication with a waste air cleaning installation, then further dried at 50° C. and under a slight vacuum of about 60 kPa and then finally dried to a constant weight under normal pressure and at about 100° C. The result obtained is powder leads which can be used as rouge, with a matt shine and good capacity for being distributed on the skin and good durability on the skin.

### EXAMPLE 5

### Eyeshadow

| | | |
|---|---|---|
| (A) | Mica and titanium dioxide | 30.000 |
| | (C.I.-No. 77019, 77891) | |
| | Mica (C.I.-No. 77019) | 8.000 |
| | Aluminium powder (C.I.-No. 77000) | 7.000 |
| | Hexamethyldisiloxane | 5.000 |
| | Phenyldimethicone | 1.000 |
| | Phenoxyethanol | 0.500 |
| | Methylparaben | 0.150 |
| | Propylparaben | 0.050 |
| (B) | Dimethicone/vinyldimethicone copolymer | 2.000 |
| | Polyethylene (powder) | 3.800 |
| | Talc | 0.800 |
| | Kaolin | 2.000 |
| | Boron nitride | 2.500 |
| | Ultramarine Blue (C.I.-No. 77007) | 2.800 |
| | Chromium hydroxide green (C.I.-No. 77289) | 7.200 |
| (C) | C11-12 isoparaffin | 21.500 |
| | Hexamethyldisiloxane | 5.000 |
| | PEG-5 soya sterol | 0.500 |
| | PEG-20 glyceryl stearate | 0.200 |

Production is effected similarly to Example 4. The result obtained is a very shiny, turquoise-coloured eyeshadow which can be very workably applied and which has good and long adhesion on the eyelid.

### EXAMPLE 6

### Eyeshadow

| | | |
|---|---|---|
| (A) | Aluminium powder (C.I.-No. 77000) | 36.500 |
| | Mica (C.I.-No. 77019) | 11.000 |
| | C11-12 isoparaffin | 3.600 |
| | Phenoxyethanol | 0.500 |
| | Methylparaben | 0.150 |
| | Propylparaben | 0.050 |
| (B) | Carnauba (Copernicia Cerifera wax) | 4.100 |
| | Talc | 2.600 |
| | Kaolin | 3.000 |
| | Boron nitride | 1.700 |
| | Silica | 1.500 |
| (C) | Aqua | 34.000 |
| | Methylcellulose | 0.550 |
| | PEG-20 soya sterol | 0.500 |
| | PEG-40 hydrogenated castor oil | 0.250 |

Production is effected in a similar manner to Example 2. The result obtained is a light, silvery shiny eyeshadow which can be very easily and uniformly distributed on the eyelid and affords very good adhesion.

### EXAMPLE 7

### Blusher

| | | |
|---|---|---|
| (A) | Mica (C.I.-No. 77019) | 15.800 |
| | Titanium dioxide and mica | 15.200 |
| | (C.I.-No. 77891, 77019) | |
| | Polypropylene (powder) | 1.000 |
| | Boron nitride | 3.500 |
| | Dimethicone 100 cSt | 4.250 |
| | Dimethiconol | 0.750 |
| (B) | Titanium dioxide | 21.800 |
| | Red iron oxide (C.I.-No. 77491) | 4.100 |
| | Yellow iron oxide (C.I.-No. 77492) | 0.900 |
| | Black iron oxide (C.I.-No. 77499) | 0.200 |
| | Talc | 5.200 |
| (C) | Aqua | 25.400 |
| | PEG-M115 | 0.300 |
| | Disodium lauryl sulphosuccinate | 0.900 |
| | Phenoxyethanol | 0.500 |
| | Methylparaben | 0.150 |
| | Propylparaben | 0.050 |

The solid constituents of the powder phase are provided in the vessel of a diaphragm vibration mixer which permits intensive but nonetheless gentle mixing of sensitive powder constituents. Dimethicone and dimethiconol are mixed and sprayed on to the surface of the powder phase. Intensive thorough mixing is then effected for about 5 minutes, further mixing possibly has to be effected until no more lumps can be found. Thereafter the mixture is produced from phase (C) by mixing water and tenside in a suitable vessel with a propeller agitator, the polyethyleneglycol is added—possibly slight heating has to be effected in that step—and then the mixture is added to the preserving agent. The phase (B) is provided in a kneader and is briefly mixed, then the phase (C) is added, thorough kneading is effected for about 5 minutes, the phase (A) is then added thereto and kneading is continued until the result is a homogeneous extrudable mixture. The subsequent procedure is then similar to the foregoing Examples. The result obtained is a matt-shiny, light, brown-red blusher which can be well distributed on the skin and exhibits excellent adhesion.

### EXAMPLE 8

### Powder Compact

| | | |
|---|---|---|
| (A) | Mica (C.I.-No. 77019) | 18.000 |
| | Titanium dioxide and mica | 15.000 |
| | (C.I.-No. 77891, 77019) | |
| | Acrylates copolymer | 24.000 |
| (B) | Titanium dioxide | 16.500 |
| | Red iron oxide (C.I.-No. 77491) | 4.850 |
| | Yellow iron oxide (C.I.-No. 77492) | 1.300 |
| | Black iron oxide (C.I.-No. 77499) | 0.350 |
| | Talc | 5.200 |
| | Boron nitride | 4.000 |
| (C) | Aqua | 8.850 |
| | PEG-100 castor oil | 0.350 |
| | PEG-20 soya sterol | 0.550 |
| | PEG-20 glyceryl stearate | 0.350 |
| | Phenoxyethanol | 0.500 |
| | Methylparaben | 0.150 |
| | Propylparaben | 0.050 |

The parabens are separately dissolved in phenoxyethanol, the water of phase (C) is put in a vessel with a propeller agitator, the remaining constituents of the phase (C) are added, agitation is effected until a homogeneous mixture is produced and then the mixture is added to the preserving agent and the mixture is transferred into a kneader. The constituents of phase (B) are now mixed in a ploughshare mixer until a uniformly coloured mixture is produced, that mixture is added to the phase (C) in the kneader and kneading is effected until a homogeneous paste is produced. A homogeneous dispersion is produced in a separate vessel with propeller agitator, in parallel with the foregoing steps, and that dispersion is added to the mixture in the kneader. The material is now emptied on to a flat sheet and dried in a vacuum drying cabinet until the mixture still has a residual moisture content of 8 to 10% by weight. The lead portions obtained in that way are now dried in the manner already described hereinbefore at 50° C. and under a vacuum of 60 kPa, and then dried at normal pressure and at about 100° C. to constant weight. The result obtained is a matt-shiny, light-brown powder compact with very good coverage and durability, which can be very easily and uniformly distributed on the skin. The addition of suitable active substances such as salicylic acid or levulinic acid benzyl or phenethyl ester also makes it possible in that manner to produce powder compacts which can help to improve juvenile skin problems and cover pimples and other skin blemishes.

The above-described leads can be well produced in diameters of 6 to 12 mm for different cosmetic uses. When using organic colour lakes or carmine, it is also possible in that way to produce rouge and blusher, concealer and other powder pencils and to use them fitted in rotary mechanisms. Instead of the above-proposed titanated mica, it is also possible to use the commercially available mica flakes coated with coloured metal oxides such as iron oxides, chromium oxide green or chromium hydroxide green or also bismuth oxychloride flakes. It is also possible to use metals in flake form or also other non-colouring ingredients in flake form such as for example transparent mica not coloured with metal oxides, synthetic mica, fine glass flakes or the like.

As already stated in the opening part of this specification, the powder leads obtained in accordance with the specified procedures can also be offered shrink-fitted in known manner into a shrink tube, glued into wood in the usual fashion, or enclosed with a film sheathing which can be at least partially torn away, of paper, plastic film or metal film or the like, or offered and used fitted in a clamping sleeve, similar to a lead pencil extension device, or fitted into a rotary mechanism.

1-45. (canceled)

46. A process for the production of pigment-bearing and/or effect substance-bearing leads, comprising the following steps:
a) at least partially mixing pigments and/or effect substances with a fluid wetting agent so that a kneadable material is produced,
b) adding further ingredients and optionally remaining pigments and/or effect substances and optionally homogenizing the resulting material,
c) extruding the material obtained in b) to obtain extrusions of predetermined diameter, and
d) drying the extrusions to a constant weight optionally after adjustment of extrusion length.

47. A process according to claim 46, wherein the pigments and effect substances are selected from the group consisting of colour pigments, pearl pigments, mica, synthetic mica, transparent mica, mica coloured with metal oxides, fine glass flakes, flakes coloured with fluorescent dyes, mica and mixtures thereof.

48. A process according to claim 46, wherein a mixture of pigments and effect substances are used in step (a).

49. A process according to claim 48, wherein in step a) pigments and effect substances are mixed separately with a fluid wetting agent and then brought together.

50. A process according to claim 48, wherein pigment and effect substances are jointly mixed with a fluid wetting agent in step (a).

51. A process according to claim 46, wherein the fluid wetting agent is selected from the group consisting of a fluid linear siloxane, a fluid cyclic siloxane, and a fluid hydrocarbon.

52. A process according to claim 46, wherein the fluid wetting agent comprises short-chain linear dimethicones.

53. A process according to claim 46, wherein the fluid wetting agent comprises dimethicones with 2 to 8 dimethylsiloxane units.

54. A process according to claim 46, wherein the fluid wetting agent comprises a mixture of dimethylsiloxanes and dimethylphenylsiloxanes.

55. A process according to claim 46, wherein the fluid wetting agent comprises cyclic siloxanes.

56. A process according to claim 55, wherein the cyclic siloxane comprises a cyclomethicone with 3 to 8 dimethylsiloxane units.

57. A process according to claim 46, further including adding solid and/or liquid lubricants to the mixture in step a) and/or b).

58. A process according to claim 57, wherein the lubricant comprises boron nitride powder.

59. A process according to claim 57, wherein the lubricant comprises fluorinated and/or phenylated siloxanes or silicone copolymers.

60. A process according to claim 59, wherein the lubricant comprises perfluorononyldimethicone.

61. A process according to claim 59, wherein the lubricant comprises dimethicone/vinyldimethicone.

62. A process according to claim 46, wherein the further ingredients added in step (b) are selected from the group consisting of fillers, thickening agents, binding agents, extrusion aids, tensides, antioxidants, fragrances, aroma substances, therapeutic active substances, preserving agents and mixtures thereof.

63. A process according to claim 62, wherein the binding agents are selected from the group consisting of wax-like polyethylene, polyethylene powder, carnauba, candelilla wax, perfluoro PG20 glyceryl stearate, and mixtures thereof.

64. A process according to claim 46, including adding tensides to the mixture prior to extruding.

65. A process according to claim 64, wherein the tenside comprises diethylhexyl sodium sulphosuccinate.

66. A process according to claim 46, including adding to the mixture an aqueous polymer dispersion of acrylates, methacrylates, polyurethanes, polyetherurethanes, polyesters, polyethers and/or mixtures thereof prior to extruding.

67. A process according to claim 51, wherein in step a) the siloxane is added in combination with a solvent selected from the group consisting of a short-chain monovalent alcohol, an ester, ketone, and mixture thereofs.

68. A process according to claim 67, wherein the solvent is selected from the group consisting of ethanol, propan-1-ol, propan-2-ol, ethylacetate, acetone, methylethylketone, and mixtures thereof.

69. A process according to claim 46, further including cutting the extrusions to a predetermined length.

70. A process according to claim 69, wherein the cut extrusions are dried in vacuum or at a slightly elevated temperature of between 40 and 70° C.

71. A process according to claim 46, including drying the extrusions by a procedure whereby drying is firstly effected for 5 minutes to 24 hours at 40 to 70° C. and thereafter drying is effected in a period of 10 to 24 hours at a temperature of to 160° C.

72. A process according to claim 71, wherein the drying is effected for 5 minutes to 4 hours at a temperature of between and 70° C. and then drying is effected for 1 to 6 hours at to 120° C.

73. A process according to claim 46, wherein step (a) comprises a step a1) wherein only a part of the pigments and/or effect substances is mixed with the wetting agent and a step a2) wherein a further part of the pigments and/or effect substances is pretreated by a procedure whereby it is mixed with an aqueous solution of a precipitation agent for a polysaccharide, then mixed with an aqueous solution of a polysaccharide which can be precipitated therewith, the mixture is dried, optionally ground, and then added to the mixture of a1).

74. A process according to claim 73, wherein the polysaccharide comprises alginic acid or pectic acid.

75. A process according to claim 73, wherein the precipitation agent comprises a soluble calcium salt.

76. A process according to claim 73, wherein the precipitation agent comprises calcium chloride.

77. A process according to claim 73, including drying the mixture of step a2) to a residual moisture content of 35 to 60% by weight.

78. A process according to claim 77, wherein the residual moisture content of the mixture of step a2) is adjusted by filtration.

79. A process according to claim 77, wherein the residual moisture content is adjusted by drying at 40° C.

80. A process according to claim 46, wherein the pigments and effect substances are used in a proportion of 20 to 80% by weight with respect to the weight of the dried extrusion.

81. A process according to claim 46, wherein the pigments and effect substances are used in a proportion of 40 to 75% by weight with respect to the dried extrusion.

82. A process according to claim 46, wherein, after step d), the dried extrusion is encased with a shrink tube.

83. A pigment-bearing and/or effect substance-bearing lead made in accordance with the process of claim 46.

84. A lead according to claim 83, wherein the lead has a diameter of 4 to 20 mm.

85. A lead according to claim 84, wherein the diameter is 6 to 12 mm.

86. A lead according to claim 83, wherein the lead comprises a marbled powder lead.

87. A lead according to claim 83, wherein the lead comprises therapeutic active substances.

88. A lead according to claim 87, wherein the lead comprises salicylic acid or levulinic acid benzyl or phenethyl ester as an active substance.
